(19) United States
(12) Patent Application Publication
(10) Pub. No.: US 2004/0162590 A1
(43) Pub. Date: Aug. 19, 2004

(54) Fully implantable miniature neurostimulator for intercostal nerve stimulation as a therapy for angina pectoris

(76) Inventors: Todd K. Whitehurst, Santa Clarita, CA (US); James P. McGivern, Stevenson Ranch, CA (US); Kelly H. McClure, Simi Valley, CA (US)

Correspondence Address: ADVANCED BIONICS CORPORATION, 25129 RYE CANYON ROAD, VALENCIA, CA, 91355, US

(21) Appl. No.: 10/731,551

(22) Filed: Dec. 8, 2003

Related U.S. Application Data
(60) Provisional application No. 60/435,019, filed on Dec. 19, 2002.

(51) Int. Cl. A61N 1/20

(52) U.S. Cl. 607/17

(57) ABSTRACT

Methods for treatment of angina pectoris (e.g., control of angina pectoris and/or relief from its symptoms) include implantation of a miniature stimulator adjacent at least one tissue influencing angina pectoris. Stimulation sites include the thoracic cardiac nerves; sympathetic ganglia at T1-T4; stellate ganglia; fat pads of the sinoatrial node, atrioventricular node, and ventricles; sympathetic trunk at T1-T4 and branches thereof; thoracic spinal nerves and branches thereof, including intercostal nerves; and the subcostal nerves. Stimulation parameters are tailored for the stimulation site. In addition, the strength and/or duration of electrical stimulation required to produce a desired therapeutic effect may be determined based on a sensed response to and/or need for treatment. Thus, the stimulation parameters may be adjusted based on a sensed condition.

The present application claims the benefit of U.S. Provisional Patent Application Serial No. 60/435,019, filed Dec. 19, 2002, which application is incorporated herein by reference in its entirety.

### BACKGROUND OF THE INVENTION

Coronary artery disease (CAD) caused over 450,000 deaths in 1997 and is the single leading cause of death in America today. Approximately 12 million Americans have a history of myocardial infarction (MI, i.e., heart attack), angina pectoris, or both. In 2002, an estimated 1.1 million Americans will have a new or recurrent MI, and more than 40 percent will die as a result. The American Heart Association estimates the annual cost of treating CAD to be about $118.2 billion.

The major symptoms of CAD include angina pectoris and MI. Angina may be described as a discomfort, a heaviness, or a pressure in the chest. It may also be described as an aching, burning, or squeezing pain. Angina is usually felt in the chest, but may also be felt in the left shoulder, arms, neck, throat, jaw, or back.

Oxygen demand that exceeds coronary vessels' capacity can cause localized ischemia. When tissue becomes ischemic, loss of function occurs within minutes. Transient ischemia causes reversible changes at the cellular and tissue level. Lack of oxygen causes a shift from aerobic to anaerobic metabolism, which increases lactic acid production, decreases cellular pH, and increases hydrogen ion concentration. Left ventricular function is impaired, causing incomplete emptying on systole, which in turn decreases cardiac output and increases left ventricular end diastolic pressure. This may lead to increased heart rate and blood pressure (hypertension), prior to the onset of pain. This cardiovascular response is a sympathetic compensation in response to the depression of myocardial function. With pain, there is also an increase in catecholamine release. Ischemic attacks subside within minutes if the imbalance between myocyte (a.k.a., cardiac cells) supply and demand for oxygen is corrected.

As is well known in the art, the electrocardiogram (ECG) signs of impending, evolving, and completed infarction follow a course from peaked T waves to elevated ST segments, to development of Q waves, to development of T wave inversion and resolution of ST segment elevation. The abnormalities to look for are “significant” Q waves, loss of precordial R height, ST elevation in contiguous leads, and T wave peaking or inversion. Any combination of these ECG abnormalities can be present during the evolution of infarction.

Prolonged cardiac ischemia (i.e., more than 30-40 minutes) causes irreversible cellular damage and necrosis, loss of myocardial contraction, and alteration of action potential conduction. Myocardial infarction (MI) is ischemic death of myocardial tissue associated with obstruction of a coronary vessel. This myocardial area of infarction becomes necrotic due to an absolute lack of blood flow. The necrotic cells are inactive electrically and their cell membranes rupture, releasing their cellular contents into the interstitial spaces. Potassium release by these cells interferes with the electrical activity of surrounding cells and leads to arrhythmias (usually premature ventricular contractions (PVCs)).

Most episodes of myocardial ischemia leading to an acute MI occur in the early morning hours. This may be related to diurnal rhythms of catecholamines and cortisol levels as well as enhanced platelet aggregation.

A narrowed vessel may develop collateral circulation. That is, small capillary-like branches of the artery may form over time in response to narrowed coronary arteries. The collaterals “bypass” the area of narrowing and help to restore blood flow. However, during times of increased exertion, the collaterals may not be able to supply enough oxygen-rich blood to the heart muscle.

Existing treatments for angina suffer from a variety of disadvantages. Currently used medications tend to improve blood circulation (i.e., oxygen supply) to the heart only acutely, if at all. (Vasodilators can improve blood supply somewhat.) Existing surgical procedures are invasive, have high morbidity, and/or are often only temporarily beneficial. What is needed are less invasive systems and methods to effectively and efficiently deliver electrical stimulation to appropriate treatment sites to treat angina and relieve patients of its symptoms.

### BRIEF SUMMARY OF THE INVENTION

The invention disclosed and claimed herein provides treatments for angina pectoris and/or for relieving its symptoms using one or more implantable microstimulators for delivering electrical stimulation. The present invention overcomes the shortfalls of all prior art treatment devices by delivering such electrical stimulation to relatively easily accessible peripheral and/or visceral nerves via a miniature stimulator implanted via a minimally invasive surgical procedure.

The stimulator used with the present invention possesses one or more of the following properties, among other properties:

at least one electrode for applying stimulating current to surrounding tissue;

electronic and/or mechanical components encapsulated in a hermetic package made from biocompatible material(s);

an electrical coil or other means of receiving energy and/or information inside the package, which receives power and/or data by inductive or radio-frequency (RF) coupling to a transmitting coil placed outside the body, thus avoiding the need for electrical leads to connect devices to a central implanted or external controller;

means for receiving and/or transmitting signals via telemetry;

means for receiving and/or storing electrical power within the stimulator; and

a form factor making the stimulator implantable via a minimally invasive procedure in a target area in the body.

A stimulator may operate independently, or in a coordinated manner with other implanted stimulators, other implanted devices, and/or with devices external to a patient's body. For instance, a stimulator may incorporate means for sensing a patient's condition, e.g., a means for sensing angina. Sensed information may be used to control the electrical stimulation parameters in a closed loop manner. The sensing and stimulating means may be incorporated into a single stimulator, or a sensing means may communicate sensed information to at least one stimulator with stimulating means.

For most patients, a continuous or intermittent stimulation throughout the day is needed to provide an adequate amount of treatment. These patients may best utilize a stimulator that has a self-contained power source sufficient to deliver repeated pulses for at least several days and that can be recharged repeatedly, if necessary. In accordance with the teachings of the present invention, the use of a stimulator with a rechargeable battery thus provides these patients the portability needed to free the patient from reliance on RF power delivery. Alternatively, the power source may be a primary battery that may last several years.

For purposes of this patent application, it is sufficient to note that RF controlled stimulators receive power and control signals from an extra corporeal antenna coil via inductive coupling of a modulated RF field. Battery-operated stimulators incorporate a power source within the device itself but rely on RF control, inductive linking, or the like to program stimulus sequences and, if a rechargeable/replenishable power source is used, to recharge/replenish the power source, when needed. In accordance with the present invention, each implanted stimulator may be commanded to produce an electrical pulse of a prescribed magnitude and duration and at a repetition rate sufficient to treat the targeted tissue.

For instance, stimulation may be initiated by start and stop commands from a patient-governed control switch or controller, which may be handheld, containing a microprocessor and appropriate nonvolatile memory, such as electronically erasable programmable read-only-memory (EEPROM). The controller may control the implantable stimulator by any of various means. For instance, the stimulator may sense the proximity of a permanent magnet located in the controller, or may sense RF transmissions from the controller.

### BRIEF DESCRIPTION OF THE DRAWINGS

The above and other aspects of the present invention will be more apparent from the following more particular description thereof, presented in conjunction with the following drawings wherein:

FIG. 1 is a schematic of the innervation of the heart;

FIG. 2A depicts nerve pathways in and near the thoracic spinal cord;

FIG. 2B depicts a section through a vertebra;

FIG. 3 depicts a typical thoracic intercostal nerve and its branches;

FIG. 4 illustrates an exemplary embodiment of a stimulation system of the present invention;

FIG. 5 illustrates exemplary external components of the invention; and

FIG. 6 depicts a system of implantable devices that communicate with each other and/or with external control/programming devices.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

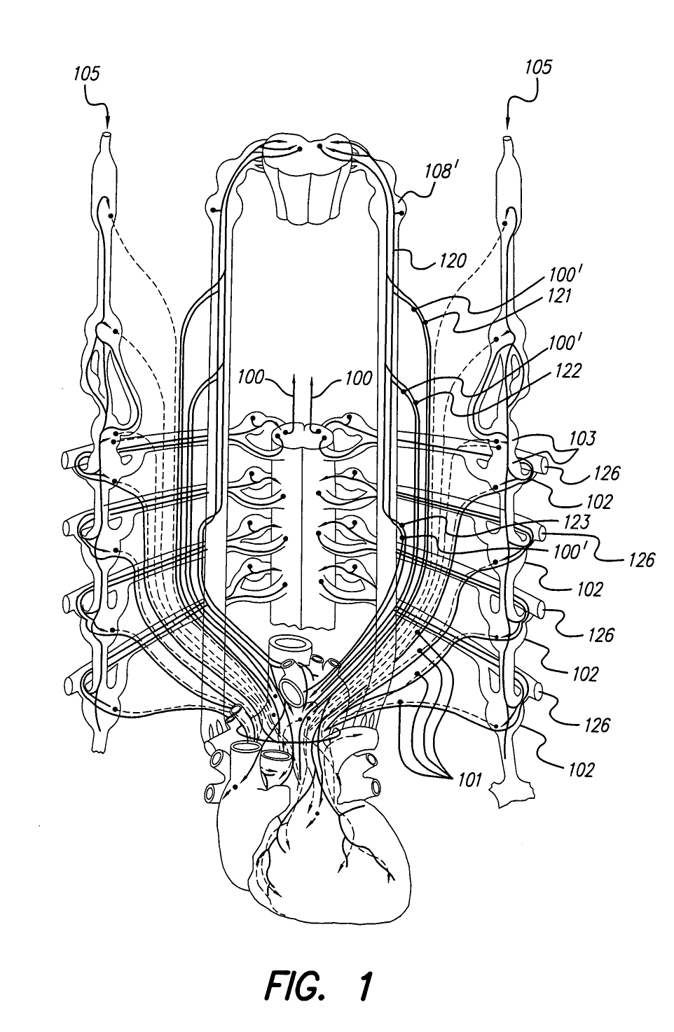

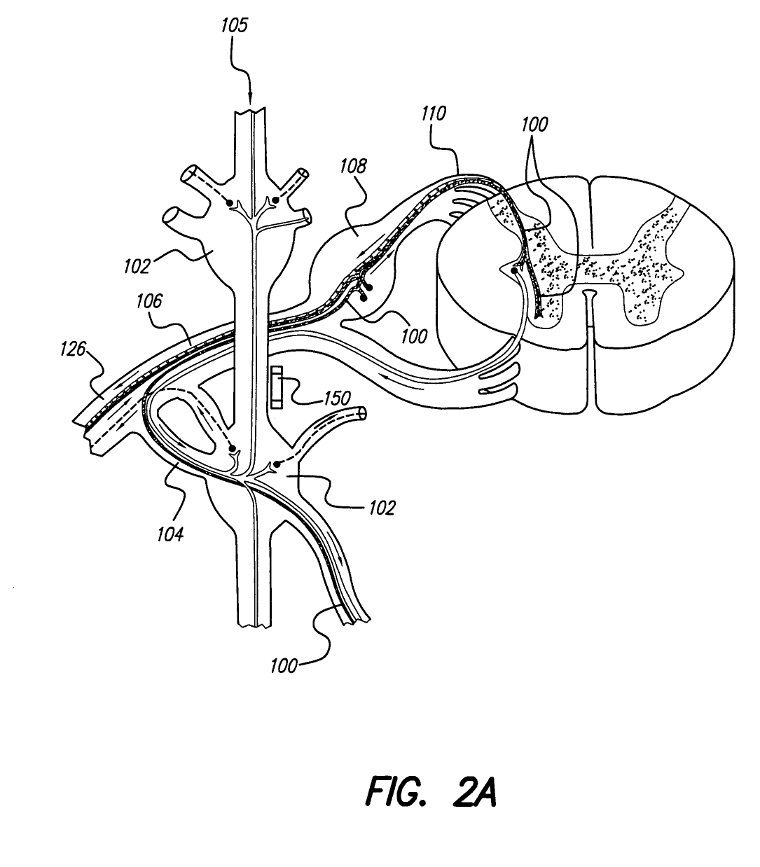

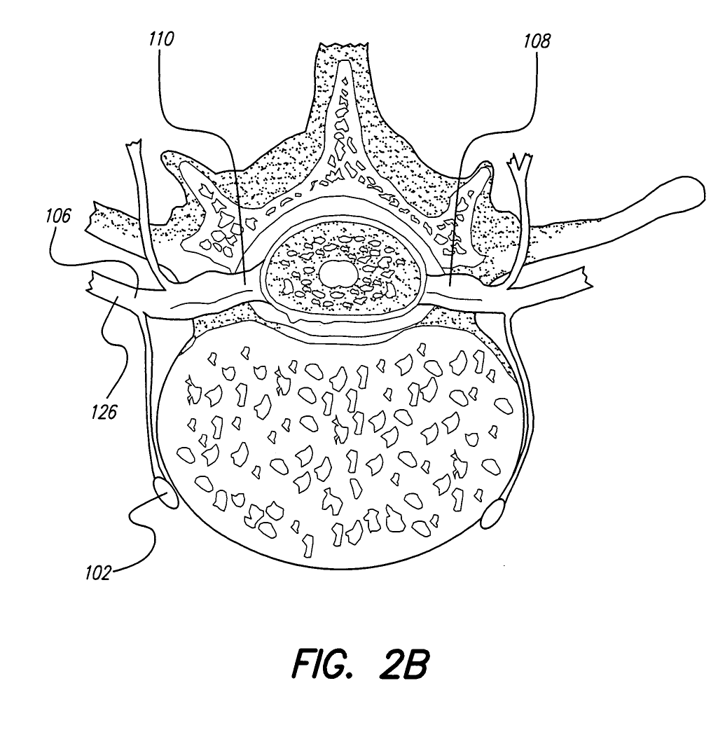

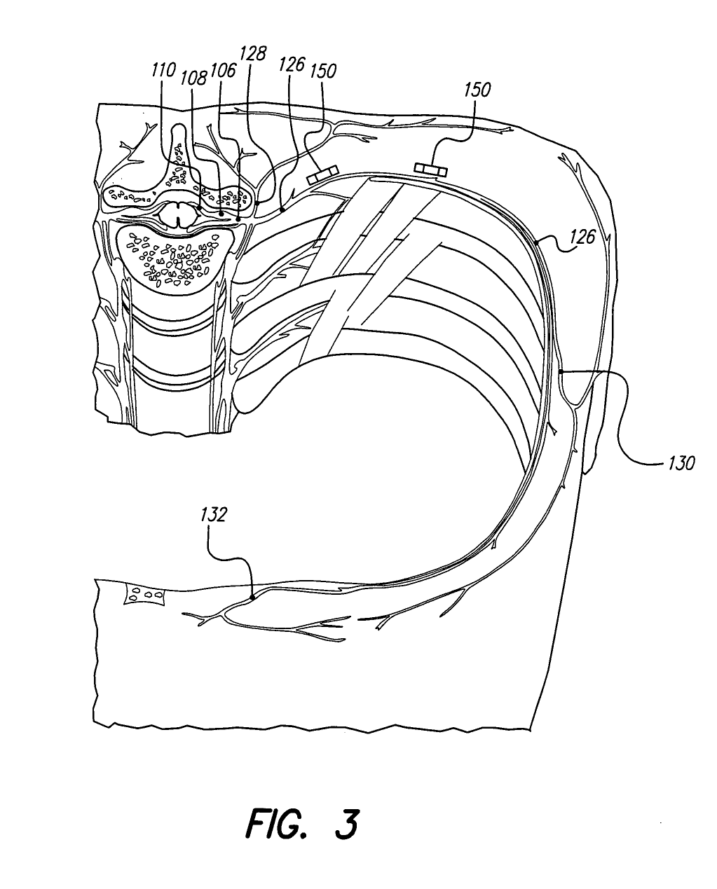

As stated above, the innervation of the heart is shown in FIG. 1. FIG. 2A shows nerve pathways in and near the thoracic part of the spinal cord. FIG. 2B shows a section through a vertebra. FIG. 3 depicts a thoracic intercostal nerve and its branches.

### Cardiac Innervation and Angina Pectoris

The inventors believe that the sensation of angina pectoris involves the activation of afferent nerve pathway(s) 100: The afferent neural messages that are interpreted by the brain as angina pectoris reach the central nervous system at least in part by traveling along visceral afferent fibers 100 that course along with cardiac sympathetic nerve fibers 101. These afferent fibers 100 have their cell bodies in the dorsal root ganglia 108 at their respective spinal levels, T1, T2, T3, and T4, with dendrites extending from the heart to these cell bodies. Signals are thus carried via these afferent fibers 100 from the heart, to and through the first through fourth thoracic sympathetic ganglia 102 of the sympathetic trunk 105, respectively. (Note that the first thoracic sympathetic ganglion 102 comprise a portion of the cervicothoracic ganglion 103, also known as the stellate ganglion 103. ) The signals then travel along the white ramus communicans 104 and spinal nerve 106, through their respective dorsal root ganglia 108, along posterior (dorsal) root 110, and into the spinal cord. The visceral afferent signals ascend the spinal cord to the brain.

The afferent neural messages that are interpreted by the brain as angina pectoris may also reach the central nervous system in part by traveling along afferent nerve pathways that course along with cardiac parasympathetic nerve fibers, i.e., the angina signal may also travel along afferent fibers 100′ that course along with parasympathetic nerve fibers arising from the vagus nerve 120. These afferent fibers 100′ have their cell bodies in the inferior vagal ganglia 108′, with dendrites extending from the heart to these cell bodies along the superior cervical (vagal) cardiac nerves 121 and the inferior cervical (vagal) cardiac nerves 122, which branch off vagus nerve 120 at the level of the cervical spinal cord, and along the thoracic cardiac branch of the vagus nerve 123, which branches off vagus nerve 120 at the level of the thoracic spinal cord. From inferior vagal ganglia 108′, afferent fibers 100′ travel to the nucleus of solitary tract of the medulla oblongata, near the posterior nucleus of the vagus nerve.

The nerve cell bodies and axons of the vagus nerve are preganglionic parasympathetic fibers. They terminate not on the heart muscle itself but on very small postganglionic parasympathetic neurons lying in small fat pads that are located next to the sinoatrial (SA) node, the atrioventricular (AV) node, and on the ventricles. These postganglionic parasympathetic cells inhibit these structures, causing a slowing of the heart rate, an increase in AV conduction time and a decreased contractility of the ventricular muscle. The parasympathetic ganglia lying in the cardiac fat pads and on the ventricles are also associated with cardiac interneurons that may modify parasympathetic ganglia function and that may also influence and be influenced by the cardiac sympathetic and cardiac afferent fibers.

### Sympathetic Blockade for Treatment of Angina

In the 1930s, it was recognized by neurosurgeons performing destructive sympathectomies for angina pectoris that local anesthetic infiltration around the stellate ganglion 103 often resulted in pain relief outlasting the duration of action of the local anesthetic drug. This observation has recently been confirmed. [See Hammond, et al. “Temporary sympathectomy in refractory angina.” Heart 1999; 81(suppl):56.] Further, this is currently the subject of a large-scale randomized double-blind placebo-controlled trial funded by the British Heart Foundation.

As mentioned above, the pathogenesis of angina pain involves the activation of afferent sympathetic pathway 100 and/or 100′. A frequent and important consequence of pain (especially when severe) is the activation of sympathetic efferent fibers. The clinical image of the patient with an acute MI (i.e., cold, clammy, sweaty, anxious, tachycardic) is secondary to this adrenergic activation. Therefore, angina might be regarded as the sensory component of a maladaptive positive feedback loop.

The angina-relieving effects of sympathetic blockade might be due to interference with this maladaptive feedback loop, in a similar manner to the way in which adenosine interrupts a re-entrant tachycardia. If such a loop exists, it may partly explain chronic refractory angina and the fact that temporary interruption of this pathway has a prolonged effect on pain. Beneficial amelioration of angina can be achieved with repeated blocks. There does not appear to be any predictability in the length of time a patient remains pain-free after successive blocks.

### Calcitonin Gene-Related Peptide (CGRP)

In 1986, the action of calcitonin gene-related peptide (CGRP) on human epicardial coronary arteries was investigated by McEwan, et al. [See McEwan, et al. “Calcitonin gene-related peptide: a potent dilator of human epicardial coronary arteries.” Circulation 1986 December; 74(6):1243-7.] In six patients receiving intracoronary doses of CGRP, a dose-dependent increase in coronary arterial diameter was observed: at the highest dose, 34%, 7%, 38%, and 40% mean increase in diameter of the circumflex, proximal, mid, and distal left anterior descending arteries, respectively. It was thus concluded that CGRP has a role in the regulation of coronary vascular smooth muscle tone.

In 1993, to investigate the possible role of CGRP in the control of vasodilation in the coronary circulation, the effects of intravenous CGRP on myocardial ischemia, cardiovascular hemodynamics, and epicardial coronary artery stenosis was studied by Uren in twelve patients with angina. [See Uren, et al. “Effect of intravenous calcitonin gene related peptide on ischemia threshold and coronary stenosis severity in humans.” Cardiovasc Res 1993 August;27(8):1477-81.] It was concluded that intravenous CGRP is a systemic arterial vasodilator which dilates coronary arteries at the site of atheromatous stenoses and which delays the onset of myocardial ischemia during treadmill exercise testing in patients with chronic stable angina.

In 2000, Franco-Cereceda, et al. reported that a subpopulation of capsaicin-sensitive cardiac C-fiber afferents co-store CGRP, substance P, and neurokinin A. [See Franco-Cereceda, et al. “Potential of calcitonin gene-related peptide (CGRP) in coronary heart disease.” Pharmacology 2000 January;60(1):1-8.] CGRP exerts positive inotropic and chronotropic effects and is one of the most potent endogenous vasodilators yet discovered. A number of endogenous agents and conditions were found to cause activation of cardiac C-fiber afferents with subsequent local release of CGRP. In myocardial ischemia and its clinical manifestations of angina pectoris and MI, C-fiber afferents not only convey the sensation of pain, but there is now also evidence of a local “efferent” release of CGRP in the heart. After being released, CGRP causes coronary vasodilation and attenuates the development of MI. CGRP may thus represent an endogenous local myocardial protective substance with interesting clinical implications.

It was reported in 1998 that low pH and lactic acid perfusion evoke a reproducible and concentration-dependent outflow of CGRP from the isolated heart, and that, in the coronary vasculature, exogenous CGRP augmented post-occlusive hyperemia. [See Kallner G. “Release and effects of calcitonin gene-related peptide in myocardial ischemia.” Scand Cardiovasc J Suppl 1998;49:1-35.] In patients undergoing CABG surgery (see below), 10-20 minutes of local ischemia was associated with increased levels of CGRP in coronary sinus blood. It was concluded that local cardiac CGRP-release from capsaicin-sensitive C-fiber afferents during myocardial ischemia functions as an endogenous physiological protective response.

### Spinal Cord Stimulation (SCS) for Angina Pectoris and Peripheral Vascular Disease (PVD)

The gate theory of pain proposed by Melzack and Wall in 1965 [see Melzack R, Wall PD. “Pain mechanisms: a new theory.” Science 1965;150:971-9] led to the first spinal cord stimulator being implanted by Norman Shealy in 1967 for cancer pain. Use of SCS in angina was reported in 1984 as a chance finding in a patient who had a stimulator for another reason. [See Sandric, et al. “Clinical and electrocardiographic improvement of ischemic heart disease after spinal cord stimulation.” Acta Neurochir Suppl 1984;33:543-6.] SCS systems were first specifically implanted for intractable angina in Australia in 1987. Since then, there have been over 70 publications on SCS in refractory angina. These studies have confirmed improvement in quality of life of these patients, fewer ischemic episodes, and reduced frequency of hospital admissions. Moreover, these effects are long-lasting and are obtained at negligible risk.

Clinicians are generally concerned about the potential risks of masking myocardial ischemia with SCS. Studies have demonstrated that SCS decreases lactate production with pacing and total ischemic burden, without an increase in silent ischemia. In a study of fifty patients with coronary artery disease and severe intractable angina treated with SCS for 1-57 months, Andersen, et al. found that SCS does not mask the pain of an acute MI. [See Andersen, et al. “Does pain relief with spinal cord stimulation for angina conceal myocardial infarction 48” British Heart Journal 1994;71:419-421.] It has also been found that mortality rates in patients with SCS systems are similar to those of the general population of patients with coronary artery disease.

SCS has been demonstrated to promote local blood flow and ischemic ulcer healing in patients with peripheral vascular disease. Positron emission tomography (PET) has shown a more homogenous pattern of coronary flow following SCS in patients with myocardial ischemia but no increase in total flow. This redistribution of flow to areas that were previously ischemic may explain the increase in exercise capacity prior to the inevitable onset of angina. To date, there has been no proof of an increase in coronary flow velocity when patients undergo pacing stress with SCS. [See Norrsell, et al. “Effects of spinal cord stimulation on coronary blood flow velocity.” Coronary Artery Disease 1998;9:273-8.]

It has been proposed that SCS may alter sympathetic/parasympathetic balance, but no change in heart rate variability has been shown in a group of post-SCS patients. [See Hautvast, et al. “Effect of spinal cord stimulation on heart rate variability and myocardial ischemia in patients with chronic intractable angina pectoris—a prospective ambulatory electrocardiographic study.” Clinical Cardiology 1998;21:33-8.] However, a decrease in resting heart rate and features suggestive of a functional sympathectomy were found in 25 SCS patients without coronary disease. [See Meglio, et al. “Spinal cord stimulation affects the central mechanisms of regulation of heart rate.” Applied Neurolphysiology 1986;49:139-146.] Cerebral PET scanning of patients with an SCS system demonstrated changes in blood flow in areas that are known to be related to pain perception in angina. [See Hautvast, et al. “Relative changes in regional cerebral blood flow during spinal cord stimulation in patients with refractory angina pectoris.” European Journal of Neuroscience 1997;9:1178-83, and Rosen, et al. “Central nervous pathways mediating angina pectoris.” Lancet 1994;344:147-150.]

### SCS versus Coronary Artery Bypass Graft (CABG) Surgery for Angina Pectoris

In 1998, Mannheimer, et al. compared SCS to coronary artery bypass graft (CABG) surgery in 104 high-risk patients who were undergoing intervention for symptomatic reasons only and who had an expected increased risk of surgical complications. [See Mannheimer, et al. “Electrical stimulation versus coronary artery bypass surgery in severe angina pectoris. The ESBY study.” Circulation 1998;97:1157-63. The patients were assessed with respect to symptoms, exercise capacity, ischemic ECG changes during exercise, rate-pressure product, mortality, and cardiovascular morbidity before and six months after the operation. The study found that both groups had approximately the same significant decrease in frequency of angina attacks as well as approximately the same significant decrease in the use of short-acting nitrates. The primary aim of both treatments is to improve quality of life by reducing symptoms. In this regard, both SCS and CABG produced similar benefits. CABG produced an additional improvement in ischemia on exercise testing at six months. Eight total deaths occurred during the follow-up period: seven total in the CABG group (four perioperative) and one in the SCS group. Cerebrovascular morbidity was also lower in the SCS group. (Most patients lose approximately ten IQ points as a result of CABG surgery.)

In a retrospective analysis of 19 patients implanted with SCS systems between 1987 and 1997, Murray, et al. found that the annual admission rate after CABG surgery was 0.97 per patient per year, compared with 0.27 after SCS. [See Murray, et al. “Spinal cord stimulation significantly decreases the need for acute hospital admission for chest pain in patients with refractory angina pectoris.” Heart 1999 July;82(1):89-92.] The mean hospital time per patient per year after CABG was 8.3 days versus 2.5 days after SCS. No unexplained ECG changes were observed during follow-up, and SCS patients presented with unstable angina and acute Ml in the usual way. The study concludes that SCS effectively prevents hospital admissions in patients with refractory angina without masking serious ischemic symptoms or leading to silent infarction.

### SCS Electrode Location and Stimulation Parameters

The electrodes for SCS for angina pectoris are typically implanted in the epidural space of the low cervical and high thoracic spinal segments, i.e., C7, T1, and T2. The stimulation voltage employed ranges from 0.7 to 9.5 volts (mean 4.2-4.5 volts), with an impedance range from 560 to 1667 Ω (mean 821-920 Ω). The stimulation frequency is typically set to 85 pps, although some studies have used frequencies as low as 20 pps with some efficacy. The pulse width typically used is 210 μsec. Intermittent stimulation is generally used. Typically the device is activated episodically by the patient, in response to anginal pain; studies have found the device active only 10-15% of a given week. [See, e.g., DeJongste, et al. “Stimulation characteristics, complications, and efficacy of spinal cord stimulation systems in patients with refractory angina: a prospective feasibility study.” Pacing and Clinical Electrophysiology 1994 November;17(11 Pt 1):1751-60, and Jessurun, et al. “Longevity and costs of spinal cord stimulation systems in patients with refractory angina pectoris.” Third Annual Svmposium on Pacing Leads, Ferrara, Italy, September 11-13, 1997.]

### Transmyocardial Revascularization Surgery

Transmyocardial revascularization (TMR) is a procedure designed to relieve severe angina in patients who are not candidates for bypass surgery or angioplasty. During TMR, a surgeon uses a laser to drill a series of holes from the outside of the heart into the heart's pumping chamber. Twenty to forty 1 mm laser channels are created during the procedure. Bleeding from the channels stops after a few minutes of pressure from the surgeon's finger. In some patients TMR is combined with bypass surgery. How TMR reduces angina still isn't fully understood. The laser may stimulate new blood vessels to grow (angiogenesis). It may destroy nerve fibers to the heart, making patients unable to feel their chest pain. In some cases, the channels may remain open, which would let oxygen-rich blood from the pumping chamber flow into the channel and then into the heart muscle.

TMR is FDA approved for use in patients with severe angina who have no other treatment options. It has also produced early promising results in three large multi-center clinical trials. The angina of 80-90 percent of patients who had this procedure has significantly improved (at least 50 percent) through one year after surgery. There's still limited follow-up data as to how long this procedure might last, however.

### Sensing Cardiac Function

A number of means are available for assessing cardiac function. An ultrasound echocardiogram can non-invasively assess a number of parameters of the heart, such as left ventricle size and cardiac output. An electrocardiogram (ECG) may be recorded non-invasively or invasively, and may be used to detect or diagnose a number of cardiac conditions, e.g., ischemia, arrhythmia, etc. Invasive pressure transducers may be used to determine left ventricular end diastolic pressure, pulmonary capillary wedge pressure, and systemic blood pressure. For instance, a thermal dilution catheter, the dye-dilution method, and/or catheter pressure transducers/catheter tip transducers may be used to measure blood pressure or cardiac output. Cardiac output, the total volume of blood pumped by the ventricle per minute, is the product of heart rate and stoke volume.

In a 1990 study of 21 heart transplant patients, Pepke-Zaba, et al. compared cardiac output measured by thermodilution and by impedance cardiography. They found close agreement between the measurements, both at rest and during exercise. Both measurements followed changes in heart rate and oxygen consumption. Both thermodilution and impedance cardiography methods elicited good reproducibility of cardiac output measurements at rest and during exercise. The authors concluded that the noninvasive and continuous record of cardiac output obtained by impedance cardiography can be used for the monitoring of cardiac output.

The inventors know of no device currently available to provide stimulation to any of the peripheral or visceral nerves associated with angina pectoris or with control of angina pain. This invention provides a means of chronically stimulating such a peripheral nerve(s) or visceral nerve(s) with a miniature implantable neurostimulator that can be implanted with a minimal surgical procedure.

Traditional SCS systems are limited to positioning in the epidural space of the spinal cord. Other limitations of traditional SCS systems include the bulky implantable pulse generator (IPG), limited life of an IPG with a primary battery, and the inconvenience of an RF powered system, among other limitations. Also, the procedure for implanting a traditional SCS system involves major surgery, with multiple incisions, local and general anesthetic, the risks of infection and other complications, and lengthy recovery time associated therewith.

According to the present invention, a miniature implantable neurostimulator, such as a bionic neuron (i.e., BION®) may be implanted via a minimal surgical procedure (e.g., via a small incision and through a cannula, endoscopically, laparoscopically) adjacent to a peripheral and/or visceral nerve(s), such as an intercostal nerve or nerve branch, associated with angina pectoris or with control of angina pain. Such a stimulator may treat angina pectoris and/or the symptoms thereof. A more complicated surgical procedure, such as a laminectomy, may be required for sufficient access to a targeted nerve fiber(s), or for fixing the neurostimulator in place.

### Stimulation of Sympathetic Fibers

For the treatment of angina pectoris (e.g., control of angina pectoris and/or relief of symptoms thereof), according to the present invention, the target site(s) of electrical stimulation include the afferent fibers 100 that course along with the cardiac sympathetic nerves 101 that exit the spinal cord at spinal levels T1, T2, T3, and T4, i.e., the thoracic (sympathetic) cardiac nerves 101. The target site(s) of electrical stimulation also include other neural tissue proximal to these nerves, i.e., the first through the fourth thoracic sympathetic ganglia 102, and stellate ganglia 103. The target site(s) of electrical stimulation also include the afferent fibers 100′ that course along with the cardiac parasympathetic nerve fibers, i.e., the superior cervical (vagal) cardiac nerve 121, the inferior cervical (vagal) cardiac nerve 122, and the thoracic cardiac branch of the vagus nerve 123. The target site(s) of electrical stimulation also include the parasympathetic ganglia and neurons lying in small fat pads that are located next to the sinoatrial (SA) node and atrioventricular (AV) node and on the ventricles. The stimulation parameters that are likely to be efficacious may be the same as the parameters used for SCS, including a stimulation frequency of about 10-85 pps. These visceral sensory fibers are likely to respond maximally to excitatory stimulation, i.e., relatively low frequency stimulation of less than about 50-100 Hz.

Electrical stimulation of the visceral afferent fibers 100 accompanying sympathetic cardiac nerves 101 is likely to lead to a release of calcitonin gene-related peptide (CGRP) onto the heart. CGRP exerts positive inotropic and chronotropic effects and is a potent endogenous vasodilators. Thus, release of CGRP may provide treatment for angina pectoris (e.g., relief of symptoms thereof).

### Stimulation of Cardiac Interneurons

Electrical stimulation of the cardiac interneurons is likely to lead to a release of CGRP onto the heart, and the associated beneficial effects stated above. To provide such stimulation, a microstimulator could be placed adjacent to the cardiac interneurons that lie in small fat pads that are located next to the sinoatrial (SA) node and atrioventricular (AV) node and on the ventricles.

### Inhibitory Stimulation of Sympathetic Fibers

Inhibitory electrical stimulation applied to sympathetic trunk 105 at spinal levels T1-T4 to block sympathetic efferents and afferents would likely prove efficacious in the treatment of angina pectoris. Such stimulation may be effected by placing a microstimulator adjacent to the sympathetic trunk 105 at spinal levels T1-T4 or adjacent to any of the distal branches thereof (i.e., sympathetic nerves in the thorax, abdomen, and pelvis, such as thoracic (sympathetic) cardiac nerves). Relatively high frequency stimulation (i.e., greater than about 50-100 Hz) of any of these target site(s) may prove inhibitory and may provide treatment for angina pectoris (e.g., relief of symptoms thereof).

### Stimulation of Somatic Nerve Fibers of Thoracic Spinal Nerves, Subcostal Nerve, and Intercostal Nerves

The intercostal nerves 126, e.g., at T1-T4, are relatively easily accessed adjacent to the ribs (FIG. 3), and these peripheral nerves may provide some treatment or control of angina, as per the gate control theory of pain. That is, since the relatively large diameter somatic sensory nerve fibers of intercostal nerves 126 enter the spinal cord at the same level as the afferent fibers 100 accompanying sympathetic cardiac nerves 101, electrical stimulation of the relatively large diameter non-nociceptive fibers of intercostal nerve(s) 126 may provide treatment (e.g., control of angina pectoris and/or relief of symptoms thereof), as per the gate theory of pain control. Similarly, the relatively large diameter somatic sensory nerve fibers of the thoracic spinal nerves 106 and/or its other branches, and/or subcostal nerve (not shown) may be stimulated to provide relief as per the gate control theory. Excitatory stimulation of relatively low frequency (e.g., less than about 50-100 Hz) and/or relatively low amplitude (e.g., less than about 15 mA) stimulation is likely to lead to the activation of the relatively large diameter non-nociceptive sensory fibers of these nerves because larger diameter fibers have a relatively lower threshold of activation than smaller diameter fibers.

As indicated above, the present invention is directed to treating angina using one or more small, implantable neurostimulators, referred to herein as “microstimulators”. The microstimulators of the present invention are preferably similar to or of the type referred to as Bionic Neuron (also referred to as a BION® microstimulator) devices. The following documents describe various details associated with the manufacture, operation, and use of BION implantable microstimulators, and are all incorporated herein by reference:

| Application/Patent/ | Filing/Publication | |
|---|---|---|
| Publication No. | Date | Title |
| U.S. Pat. No. 5,193,539 | Issued | Implantable Microstimulator |
| | Mar. 16, 1993 | |
| U.S. Pat. No. 5,193,540 | Issued | Structure and Method of Manufacture of an Implantable |
| | Mar. 16, 1993 | Microstimulator |
| U.S. Pat. No. 5,312,439 | Issued | Implantable Device Having an Electrolytic Storage |
| | May 17, 1994 | Electrode |
| PCT Publication | published | Battery-Powered Patient Implantable Device |
| WO 98/37926 | Sep. 3, 1998 | |
| PCT Publication | published | System of Implantable Devices For Monitoring and/or |
| WO 98/43700 | Oct. 8, 1998 | Affecting Body Parameters |
| PCT Publication | published | System of Implantable Devices For Monitoring and/or |
| WO 98/43701 | Oct. 8, 1998 | Affecting Body Parameters |
| U.S. Pat. No. 6,051,017 | Issued | Improved Implantable Microstimulator and Systems |
| | Apr. 18, 2000 | Employing Same |
| | published | Micromodular Implants to Provide Electrical Stimulation |
| | September, 1997 | of Paralyzed Muscles and Limbs, by Cameron, et al., |
| | | published in IEEE Transactions on Biomedical |
| | | Engineering, Vol. 44, No. 9, pages 781-790. |

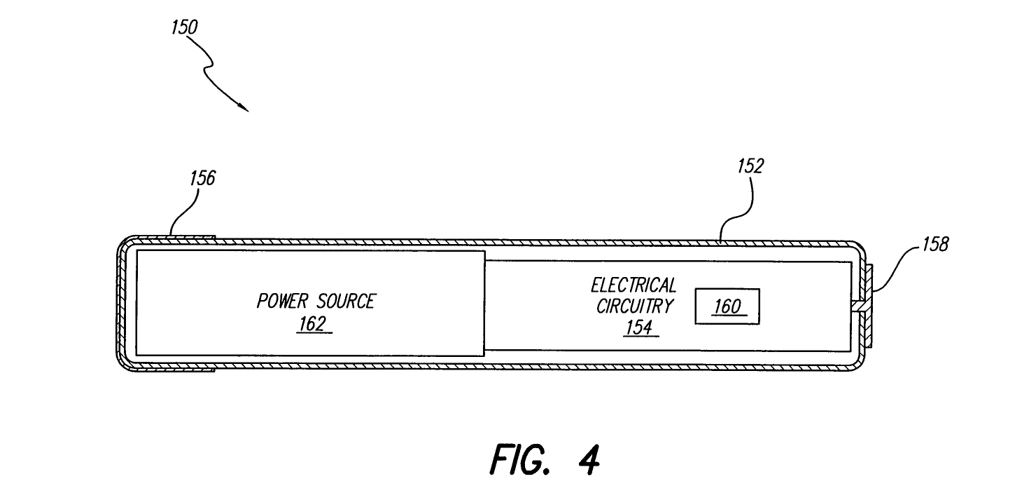

To treat angina pectoris, a microminiature stimulator 150, such as a BION microstimulator, illustrated, e.g., in FIGS. 3 and 4, is preferably implanted, e.g., adjacent to an intercostal nerve 126. For instance, the microstimulator may be placed between two ribs, preferably on the left between T1 and T2 (or T2 and T3), for stimulation of intercostal nerve 126 at Ti (or T2, respectively).

Based on the gate control theory described earlier, stimulating fast-conducting, larger diameter nerve fibers will block, or gate, the slower pain signals from reaching the brain. The somatic sensory fibers responsible for touch, pressure, and position sense are carried through relatively large diameter nerve fibers (i.e., A-α and/or A-β fibers), while smaller diameter nerve fibers (e.g., A-δ and/or C fibers) carry pain signals. As such, angina pectoris may be treated with stimulation additionally or alternatively applied to the larger diameter nerve fibers, which larger diameter fibers have a relatively lower threshold of activation than smaller diameter fibers. Excitatory stimulation of relatively low frequency (e.g., less than about 50-100 Hz) and/or relatively low amplitude (e.g., less than about 15 mA) stimulation is likely to lead to the activation of these relatively large diameter non-nociceptive sensory fibers.

In accordance with the present invention, a single microstimulator 150 may be implanted, or two or more microstimulators may be implanted to achieve greater stimulation of the targeted tissue, or for a longer period of time. In the example of FIG. 4, microstimulator device 150 includes a narrow, elongated case 152 containing electronic circuitry 154 connected to electrodes 156 and 158, which may pass through the walls of the case at either end. Alternatively, electrodes 156 and/or 158 may be built into and/or onto the case and/or arranged on a distal portion of a lead, as described below. As detailed in the referenced publications, electrodes 156 and 158 generally comprise a stimulating electrode (to be placed close to the nerve) and an indifferent electrode (for completing the circuit). Other configurations of microstimulator device 150 are possible, as is evident from the above-referenced publications.

A preferred implantable microstimulator 150 is sufficiently small to permit its placement near the structures to be stimulated. (As used herein, “adjacent” and “near” mean as close as reasonably possible to target tissue(s), including touching or even being positioned within the tissue, but in general, may be as far as can be reached with the stimulation pulses.) As such, case 152 may have a diameter of about 4-5 mm, or only about 3 mm, or even less than about 3 mm. In these configurations, case length may be about 25-35 mm, or only about 20-25 mm, or even less than about 20 mm. The shape of the microstimulator may be determined by the structure of the desired target, the surrounding area, and the method of implantation. A thin, elongated cylinder with electrodes at the ends, as shown in FIG. 4, is one possible configuration, but other shapes, such as rounded cylinders, spheres, disks, and helical structures, are possible, as are different configurations of and/or additional electrodes.

Microstimulator 150 is preferably implanted with a surgical insertion tool specially designed for the purpose (see, e.g., U.S. Pat. No. 6,582,441), or may be placed, for instance, via a small incision and through a small cannula. Alternatively, device 150 may be implanted via conventional surgical methods, or may be inserted using other endoscopic or laparoscopic techniques. A more complicated surgical procedure may be required for purposes of fixing the microstimulator in place.

The external surfaces of stimulator 150 are advantageously composed of biocompatible materials. To protect the electrical components inside stimulator 150, at least a portion of case 152 is hermetically sealed. For instance, stimulator case 152 may be made of, for instance, glass, ceramic, or other material that provides a hermetic package that excludes water vapor but permits passage of electromagnetic fields used to transmit data and/or power. For additional protection against, e.g., impact, the case may be made of metal (e.g., titanium) or ceramic, which materials are also, advantageously, biocompatible. In addition, stimulator 150 may be configured to be Magnetic Resonance Imaging (MRI) compatible. Electrodes 156 and 158 may be made of a noble or refractory metal or compound, such as platinum, iridium, tantalum, titanium, titanium nitride, niobium, or alloys of any of these, in order to avoid corrosion or electrolysis, which could damage the surrounding tissues and the device.

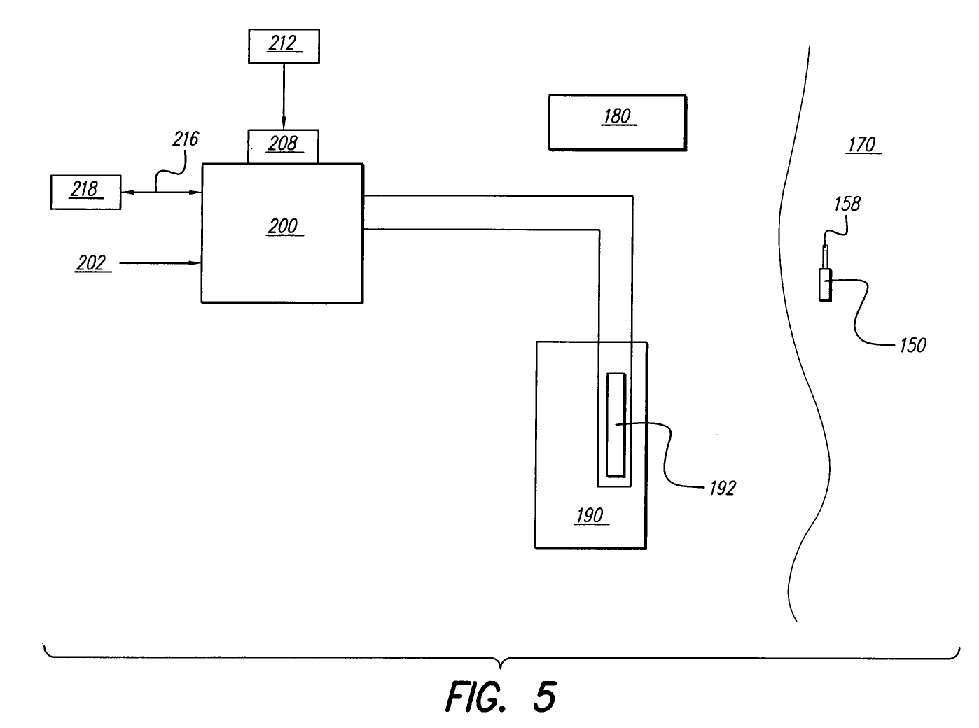

In some embodiments of the instant invention, microstimulator 150 comprises at least one, leadless electrode. However, one, some, or all electrodes may alternatively be located at the end of short, flexible leads (e.g., see FIG. 5) as described in U.S. patent application Ser. No. 09/624,130, filed Jul. 24, 2000 (which claims priority to U.S. Provisional Patent Application No. 60/156,980, filed Oct. 1, 1999), which is incorporated herein by reference in its entirety. Other configurations may also permit electrical stimulation to be directed more locally to specific tissue a short distance from the surgical fixation of the bulk of the implantable stimulator 150, while allowing elements of stimulator 150 to be located in a more surgically convenient site. Such configurations minimize the distance traversed and the surgical planes crossed by the device and any lead(s). In most embodiments, the leads are no longer than about 150 mm.

Microstimulator 150 contains, when necessary and/or desired, electronic circuitry 154 (FIG. 4) for receiving data and/or power from outside the body by inductive, radio-frequency (RF), or other electromagnetic coupling. In some embodiments, electronic circuitry 154 includes an inductive coil for receiving and transmitting RF data and/or power, an integrated circuit (IC) chip for decoding and storing stimulation parameters and generating stimulation pulses (either intermittent or continuous), and additional discrete components required to complete the circuit functions, e.g. capacitor(s), resistor(s), coil(s), and the like. Circuitry 154 may dictate, for instance, the amplitude and duration of the electrical current pulses.

Microstimulator 150 also includes, when necessary and/or desired, a programmable memory 160 for storing set(s) of data, stimulation, and/or control parameters. Among other things, memory 164 may allows stimulation and/or control parameters to be adjusted to settings that are safe and efficacious with minimal discomfort for each individual. Specific parameters may provide therapeutic advantages for different patients or for various types and classes of angina pectoris. For instance, some patients may respond favorably to intermittent stimulation, while others may require continuous stimulation for treatment and relief.

In addition, different parameters may have different effects on different tissue. Therefore, stimulation and control parameters may be chosen to target specific neural or other cell populations and/or to exclude others, or to increase activity in specific neural or other cell populations and/or to decrease activity in others. For example, relatively low frequency neurostimulation (i.e., less than about 50-100 Hz) may have an excitatory effect on surrounding neural tissue, leading to increased neural activity (“excitatory stimulation”), whereas relatively high frequency neurostimulation (i.e., greater than about 50-100 Hz) may have an inhibitory effect, leading to decreased neural activity (“inhibitory stimulation”). As another example, relatively low levels of stimulation current (typically less than about 15 mA, but dependent on the distance between electrodes and nerve fibers) are likely to recruit only relatively large diameter fibers (e.g., A-α and/or A-β fibers), while nociceptive fibers are typically relatively small diameter fibers (e.g., A-δ and/or C fibers).

Some embodiments of implantable stimulator 150 also includes a power source and/or power storage device 162 (FIG. 4). Possible power options, described in more detail below, include but are not limited to an external power source coupled to stimulator 150 (e.g., via an RF link), a self-contained power source utilizing any suitable means of generation or storage of energy (e.g., a primary battery, a replenishable or rechargeable battery such as a lithium ion battery, an electrolytic capacitor, a super- or ultra-capacitor, or the like), and if the self-contained power source is replenishable or rechargeable, means of replenishing or recharging the power source (e.g., an RF link, an optical link, a thermal link, or other energy-coupling link).

According to certain embodiments of the invention, a microstimulator operates independently. According to other embodiments of the invention, a microstimulator operates in a coordinated manner with other microstimulator(s), other implanted device(s), and/or other device(s) external to the patient's body. For instance, a microstimulator may control or operate under the control of another implanted microstimulator(s), other implanted device(s), or other device(s) external to the patient's body. A microstimulator may communicate with other implanted microstimulators, other implanted devices, and/or devices external to a patient's body via, e.g., an RF link, an ultrasonic link, a thermal link, or an optical link. Specifically, a microstimulator may communicate with an external remote control (e.g., patient and/or physician programmer) that is capable of sending commands and/or data to a microstimulator and that is preferably capable of receiving commands and/or data from a microstimulator.

In certain embodiments, and as illustrated in FIG. 5, the patient 170 switches stimulator 150 on and off by use of controller 180, which may be handheld. Implantable stimulator 150 may be operated by controller 180 by any of other various means, including sensing the proximity of a permanent magnet located in controller 180, sensing RF transmissions from controller 180, or the like.

Additional and alternative exemplary external components for programming and/or providing power to various embodiments of stimulator 150 are also illustrated in FIG. 5. When communication with such a stimulator 150 is desired, patient 170 is positioned on or near external appliance 190, which appliance contains one or more inductive coils 192 or other means of communication (e.g., RF transmitter and receiver). External appliance 190 is connected to or is a part of external circuitry appliance 200 which may receive power 202 from a conventional power source. External appliance 200 contains manual input means 208, e.g., a keypad, whereby the patient 170 or a caregiver 212 (e.g., a clinician) may request changes in stimulation parameters produced during the normal operation of the implantable stimulator 150. In these embodiments, manual input means 208 preferably includes various electro-mechanical switches and/or visual display devices that provide the patient and/or caregiver with information about the status and prior programming of the implantable stimulator 150.

Alternatively or additionally, external electronic appliance 200 is provided with an electronic interface means 216 for interacting with other computing means 218, such as via serial interface cable or infrared link to a personal computer or telephone modem or the like. Such interface means 216 may permit a clinician to monitor the status of the implant and prescribe new stimulation parameters from a remote location.

One or more of the external appliance(s) may be embedded in a cushion, mattress cover, garment, or the like. Other possibilities exist, including a strap, patch, or other structure(s) that may be affixed to the patient's body or clothing. External appliances may include a package that can be, e.g., worn on the belt, may include an extension to a transmission coil affixed, e.g., with a Velcro® band or an adhesive, or may be combinations of these or other structures able to perform the functions described herein.

In order to help determine the strength and/or duration of electrical stimulation required to produce the desired therapeutic effect, in some embodiments, a patient's response to and/or need for treatment is sensed, e.g., via ECG changes or via an oxygen sensor in the coronary circulation. Sensed information may be used to control the stimulation parameters of a microstimulator in a closed-loop manner. According to some embodiments of the invention, the sensing and stimulating means are both incorporated into a single microstimulator. Thus, when microstimulator 150 is implanted, for example, near the sympathetic trunk, the signals from a sensor built into microstimulator 150 may be used to adjust stimulation parameters. For instance, with stimulator 150 near the sympathetic trunk (e.g., at a level which sends afferent fibers into the spinal cord at T1-T2), stimulation may be initiated or amplitude increased if increased sympathetic activity is sensed via ENG.

According to other embodiments, the sensing means are incorporated into at least one “microstimulator” (that may or may not have stimulating means), and the sensed information is communicated to at least one other microstimulator with stimulating means. A microstimulator or other sensor may additionally or alternatively incorporate means of sensing other measures of the state of the patient, e.g., EMG, acceleration, patient activity, respiratory rate, medication levels, neurotransmitter levels, hormone levels, interleukin levels, cytokine levels, lymphokine levels, chemokine levels, growth factor levels, enzyme levels, and/or levels of other blood-borne compounds. For instance, one or more Chemically Sensitive Field-Effect Transistors (CHEMFETs), such as Enzyme-Selective Field-Effect Transistors (ENFETs) or Ion-Sensitive Field-Effect Transistors (ISFETs, as are available from Sentron CMT of Enschede, The Netherlands), may be used.

Thus, a “microstimulator” dedicated to sensory processes may communicate with a microstimulator that provides the stimulation pulses. For instance, a microstimulator, such as a BION® manufactured by Advanced Bionics of Sylmar, California, may be used to detect abnormal cardiac electrocardiogram (ECG) events. A BION may use one of many algorithms for analyzing ECGs. These algorithms can operating in the frequency domain, time domain or both. They may employ linear, non-linear, or statistical analysis to categorize the electrogram as originating from various modes, i.e., normal sinus rhythms, sinus tachycardia, ventricular tachycardia, and ventricular fibrillation. In addition, by finding the P, R, and T waves or analyzing the timing of the relationships and durations of the P-wave, QRS complex, and T-wave, it is possible to identify various disease states and make predictive diagnosis about perfusion of the myocardium. Other abnormalities that may be monitored include ST segment elevation, T wave peaking or inversion, among others discussed earlier. See, for instance, U.S. Pat. No. 5,513,644, titled “Cardiac arrhythmia detection system for an implantable stimulation device,” which is incorporated herein by reference in its entirety.

Addition possibilities include a microstimulator(s) or other sensor(s) to detect markers of ischemia, e.g., Troponin-I or Troponin-T. See, for instance, U.S. Pat. No. 5,753,517, titled “Quantitative immunochromatographic assays,” which is incorporated herein by reference in its entirety. Antibodies that bind to Troponin-I may be sensed, for instance, with a detection reagent (to which the antibodies bind) and measured using electrical conductivity or capacitance. A microstimulator or other sensor could additionally or alternatively measure an antibody that fluoresces when binding to Troponin-I, for instance, with an LED encased in a hermetic glass seal coated with the antibody.

Other methods of determining the required stimulation include an oxygen sensor in the coronary circulation, as well as other methods mentioned herein, and yet others that will be evident to those of skill in the art upon review of the present disclosure. The sensed information may be used to control the electrical and/or control parameters in a closed-loop manner.

For instance, in several embodiments of the present invention, a first and second “stimulator” are provided. The second “stimulator” periodically (e.g. once per minute) records e.g., ECG, which it transmits to the first stimulator. Implant circuitry 154 may, if necessary, amplify, filter, process, then transmit these sensed signals, which may be analog or digital. The first stimulator uses the sensed information to adjust stimulation parameters according to an algorithm programmed, e.g., by a physician. For example, amplitude of stimulation may be initiated or increased in response to ST segment elevation and/or T wave inversion. More preferably, one “microstimulator” performs the sensing, stimulation parameter adjustments, and current generating functions.

While a microstimulator may also incorporate means of sensing angina or its symptoms, it may alternatively or additionally be desirable to use a separate or specialized implantable device to sense and telemeter physiological conditions/responses in order to adjust stimulation parameters. This information may then be transmitted to an external device, such as external appliance 220, or may be transmitted directly to implanted stimulator(s) 150. However, in some cases, it may not be necessary or desired to include a sensing function or device, in which case stimulation parameters are determined and refined, for instance, by patient feedback.

Thus, it is seen that in accordance with the present invention, one or more external appliances are preferably provided to interact with microstimulator 150 to accomplish one or more of the following functions:

Function 1: If necessary, transmit electrical power from the external electronic appliance 200 via appliance 190 to the implantable stimulator 150 in order to power the device and/or recharge the power source/storage device 162. External electronic appliance 200 may include an automatic algorithm that adjusts stimulation parameters automatically whenever the implantable stimulator(s) 150 is/are recharged.

Function 2: Transmit data from external appliance 200 via external appliance 190 to implantable stimulator 150 in order to change the operational parameters (e.g., electrical stimulation parameters) used by stimulator 150.

Function 3: Transmit sensed data indicating a need for treatment or in response to stimulation (e.g., ECG) from implantable stimulator 150 to external appliance 200 via external appliance 190.

Function 4: Transmit data indicating state of the implantable stimulator 150 (e.g., battery level, stimulation settings, etc.) to external appliance 200 via external appliance 190.

By way of example, a treatment modality for angina pectoris may be carried out according to the following sequence of procedures:

1. A stimulator 150 is implanted so electrode(s) 156 and/or 158 are adjacent to the sympathetic trunk at the level at which the afferent fibers in the sympathetic trunk enter the spinal cord at predominantly levels T1 and T2.

2. Using Function 2 described above (i.e., transmitting data) of external electronic appliance 200 and external appliance 190, implantable stimulator 150 is commanded to produce a series of inhibitory electrical stimulation pulses.

3. Set stimulator on/off period to an appropriate setting, e.g., five seconds on then five seconds off.

4. After each stimulation pulse, series of pulses, or at some other predefined interval, any change in sympathetic firing rate is sensed (via ENG), preferably by one or more electrodes 156 and 158 of implantable stimulator 150. These responses are converted to data and telemetered out to external electronic appliance 200 via Function 3.

5. From the response data received at external appliance 200 from the implantable stimulator 150, or from other assessment, the stimulus threshold for obtaining a reflex response is determined and is used by a clinician acting directly 212 or by other computing means 218 to transmit the desired stimulation parameters to the implantable stimulator 150 in accordance with Function 2. Alternatively, external appliance 200 makes the proper adjustments automatically, and transmits the proper stimulation parameters to stimulator 150. In yet another alternative, stimulator 150 adjusts stimulation parameters automatically based on the sensed response.

6. When patient 170 desires to invoke an electrical stimulation to alleviate symptoms (e.g., pain, loss of function, etc.), patient 170 employs handheld controller 180 to set the implantable stimulator 150 in a state where it delivers a prescribed stimulation pattern from a predetermined range of allowable stimulation patterns.

7. Patient 170 employs controller 180 to turn off stimulator 150, if desired.

8. Periodically, the patient or caregiver recharges the power source/storage device 162 of implantable stimulator 150, if necessary, in accordance with Function 1 described above (i.e., transmit electrical power).

As another example, a treatment modality for angina pectoris may be carried out according to the following sequence of procedures:

1. A stimulator 150 is implanted so electrode(s) 156 and/or 158 are adjacent to an intercostal nerve 126. For instance, to stimulate left intercostal nerve 126 at the level of T1, a microstimulator may be placed between the two left ribs at T1 and T2. Additionally or alternatively, a microstimulator may be placed between the two left ribs at T2 and T3 to stimulate left intercostal nerve 126 at the level of T2.

2. Using Function 2 described above (i.e., transmitting data) of external electronic appliance 200 and external appliance 190, implantable stimulator 150 is commanded to produce a series of excitatory electrical stimulation pulses.

3. Set stimulator on/off period to an appropriate setting, e.g., one hour on and seven hours off.

4. After each stimulation pulse, series of pulses, or at some other predefined interval, any change in ECG is sensed, preferably by one or more electrodes 156 and 158 of implantable stimulator 150. These responses are converted to data and telemetered out to external electronic appliance 200 via Function 3.

5. From the response data received at external appliance 200 from the implantable stimulator 150, or from other assessment (e.g., based on patient's reported threshold of sensation and reported threshold of pain), the stimulus threshold for obtaining a reflex response is determined and is used by a clinician acting directly 212 or by other computing means 218 to transmit the desired stimulation parameters to the implantable stimulator 150 in accordance with Function 2. For example, the minimum stimulation level may be set at the patient's reported threshold of sensation, while the maximum stimulation level may be set at or slightly below the patient's reported threshold of pain. Alternatively, external appliance 200 makes the proper adjustments automatically, and transmits the proper stimulation parameters to stimulator 150. In yet another alternative, stimulator 150 adjusts stimulation parameters automatically based on the sensed response.

6. When patient 170 desires to invoke an electrical stimulation to alleviate symptoms (e.g., pain, loss of function, etc.), patient 170 employs handheld controller 180 to set the implantable stimulator 150 in a state where it delivers a prescribed stimulation pattern from a predetermined range of allowable stimulation patterns.

7. Patient 170 employs controller 180 to turn off stimulator 150, if desired.

8. Periodically, the patient or caregiver recharges the power source/storage device 162 of implantable stimulator 150, if necessary, in accordance with Function 1 described above (i.e., transmit electrical power).

For the treatment of any of the various types and classes of angina pectoris, it may be desirable to modify or adjust the algorithmic functions performed by the implanted and/or external components, as well as surgical approaches. For example, in some situations, it may be desirable to employ more than one implantable stimulator 150, each of which could be separately controlled by means of a digital address. Multiple channels and/or multiple patterns of stimulation might thereby be programmed by the clinician and controlled by the patient in order to, for instance, deal with complex or multidimensional pain such as may occur as a result of diffuse anginal pain requiring simultaneous stimulation of multiple areas (e.g., T1 and T2), for example.

In some embodiments discussed earlier, microstimulator 150, or two or more microstimulators, is controlled via closed-loop operation. A need for and/or response to stimulation is sensed via microstimulator 150, or by an additional microstimulator (which may or may not be dedicated to the sensing function), or by another implanted or external device. If necessary, the sensed information is transmitted to microstimulator 150. In some cases, the sensing and stimulating are performed by one stimulator. In some embodiments, the stimulation parameters used by microstimulator 150 are automatically adjusted based on the sensed information. For instance, one “microstimulator” may performs the sensing, stimulation parameter adjustments, and current generating functions. Thus, the stimulation parameters may be adjusted in a closed-loop manner to provide stimulation tailored to the need for and/or response to stimulation.

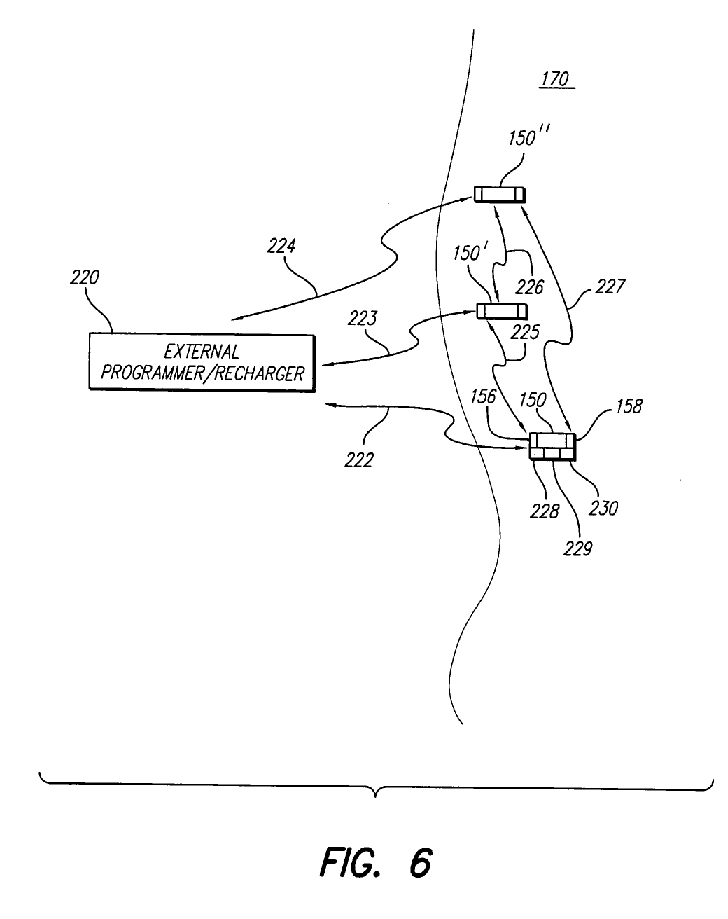

For example, as seen in FIG. 6, a first microstimulator 150, implanted in or adjacent first intercostal nerve (left), provides electrical stimulation via electrodes 156 and 158 to a first location; a second microstimulator 150′ provides electrical stimulation to a second location, e.g., second intercostal nerve (left); and a third microstimulator 150″ provides electrical stimulation to a third location, e.g., the sympathetic trunk at T1 or T2. As mentioned earlier, the implanted devices may operate independently or may operate in a coordinated manner with other similar implanted devices, other implanted devices, or other devices external to the patient's body, as shown by the control lines 222, 223 and 224 in FIG. 6. That is, in accordance with certain embodiments of the invention, an external controller 220 controls the operation of one or more of the implanted microstimulators 150, 150′ and 150″.

According to various embodiments of the invention, an implanted device, e.g., microstimulator 150, may control or operate under the control of another implanted device(s), e.g., microstimulator 150′ and/or microstimulator 150″. That is, a device made in accordance with the invention may communicate with other implanted stimulators, other implanted devices, and/or devices external to a patient's body, e.g., via an RF link, an ultrasonic link, a thermal link, an optical link, or the like. Specifically, as illustrated in FIG. 6, microstimulator 150, 150′, and/or 150″, made in accordance with the invention, may communicate with an external remote control (e.g., patient and/or physician programmer 220 and/or the like) that is capable of sending commands and/or data to implanted devices and may also be capable of receiving commands and/or data from implanted devices.

Microstimulators made in accordance with the invention further incorporate, in some embodiments, first sensing means 228 for sensing therapeutic effects, clinical variables, or other indicators of the state of the patient, such as ECG. The stimulators additionally or alternatively incorporate second means 229 for sensing, e.g., levels and/or changes in pain medication and/or other markers of the potential for angina. The stimulators additionally or alternatively incorporate third means 230 for sensing electrical current levels and/or waveforms supplied by another source of electrical energy. Sensed information may then be used to control the parameters of the stimulator(s) in a closed loop manner, as shown by control lines 225, 226, and 227. Thus, the sensing means may be incorporated into a device that also includes electrical stimulation means, or the sensing means (that may or may not have stimulating means), may communicate the sensed information to another device(s) with stimulating means.

Thus, for the treatment of angina pectoris (e.g., control of angina pectoris and/or relief of symptoms thereof), according to the present invention, the target site(s) of electrical stimulation include the afferent fibers 100 that course along with the cardiac sympathetic nerves 101 that exit the spinal cord at spinal levels T1, T2, T3, and T4, i.e., the thoracic (sympathetic) cardiac nerves 101. The target site(s) of electrical stimulation also include other neural tissue proximal to these nerves, i.e., the first through the fourth thoracic sympathetic ganglia 102, and stellate ganglia 103. The target site(s) of electrical stimulation also include the afferent fibers 100′that course along with the cardiac parasympathetic nerve fibers, i.e., the superior cervical (vagal) cardiac nerve 121, the inferior cervical (vagal) cardiac nerve 122, and the thoracic cardiac branch of the vagus nerve 123. The target site(s) of electrical stimulation also include the parasympathetic ganglia and neurons lying in small fat pads that are located next to the sinoatrial (SA) node and atrioventricular (AV) node and on the ventricles. The stimulation parameters that are likely to be efficacious may be the same as the parameters used for SCS, including a stimulation frequency of about 10-85 pps. These visceral sensory fibers are likely to respond maximally to excitatory stimulation (at a relatively low frequency, e.g., less than about 50-100 Hz).

Additionally or alternatively, to block sympathetic efferents and afferents, inhibitory electrical stimulation may be applied by a microstimulator placed adjacent to the sympathetic trunk 105 at spinal levels T1 through T4, or adjacent to any of the distal branches thereof, such as sympathetic nerves in the thorax, abdomen, and pelvis, such as thoracic (sympathetic) cardiac nerves 101. Inhibitory stimulation (at a relatively high frequency, e.g., greater than about 50-100 Hz) of any of these target site(s) may provide treatment for angina pectoris.

Electrical stimulation of the relatively large diameter non-nociceptive fibers of intercostal nerves 126, e.g., at T1-T4, other branches of thoracic spinal nerves 106, or spinal nerve(s) 106 themselves, may provide treatment (e.g., control of angina pectoris and/or relief of symptoms thereof), per the gate theory of pain control. As shown in FIG. 3, branches of the thoracic spinal nerves 106 include, in addition to intercostal nerves 126 (a.k.a. anterior (ventral) ramus of thoracic spinal nerve), the posterior (dorsal) ramus 128 of the thoracic spinal nerve and its branches, the lateral cutaneous branch 130 of the intercostal nerve and its branches, and the anterior cutaneous branch 132 of the intercostal nerve and its branches. In addition, subcostal nerve (not shown) may also/instead be stimulated to provide relief as per the gate control theory. Relatively low frequency excitatory stimulation (e.g., less than about 50-100 Hz) and/or relatively low amplitude (e.g., less than about 15 mA) stimulation is likely to lead to the activation of the relatively large diameter non-nociceptive fibers of the intercostal nerves 126.

Furthermore, sensing means described earlier may be used to orchestrate first the activation of microstimulator(s) targeting one or more nerve fibers, and then, when appropriate, the microstimulator(s) targeting nerve fibers in another area and/or by a different means. Alternatively, this orchestration may be programmed, and not based on a sensed condition. In yet another alternative, this coordination may be controlled by the patient via the patient programmer.

While the invention herein disclosed has been described by means of specific embodiments and applications thereof, numerous modifications and variations could be made thereto by those skilled in the art without departing from the scope of the invention set forth in the claims.

What is claimed is:

1. A method for treating a patient with angina pectoris, comprising:
providing a miniature leadless implantable stimulator with at least one electrode and with a size and shape suitable for placement of the entire stimulator adjacent to a nerve;
implanting the stimulator adjacent to at least one tissue influencing the angina pectoris of the patient, which tissue is at least one of an intercostal nerve and an intercostal nerve branch;
providing operating power to the stimulator;
using an external appliance to transmit stimulation parameters to the stimulator;
receiving the stimulation parameters at the stimulator;
generating stimulation pulses in accordance with the stimulation parameters, which pulses are generated by the stimulator;
delivering stimulation pulses via the stimulator to the at least one of the intercostal nerves and intercostal nerve branches influencing angina pectoris as a treatment for angina pectoris.

2. The method of claim 1 further comprising generating and delivering excitatory stimulation pulses to at least one of the intercostal nerves and the intercostal nerve branches.

3. The method of claim 1 further comprising generating and delivering stimulation pulses of less than about 15 mA to at least one of the intercostal nerves and the intercostal nerve branches.

4. The method of claim 1 wherein the implantable stimulator further comprises at least one sensor and the method further comprises sensing at least one condition of the patient.

5. The method of claim 4 wherein the at least one sensed condition is used to adjust the stimulation parameters.

6. The method of claim 5 wherein the parameter adjustment is performed using the at least one external appliance.

7. The method of claim 5 wherein the parameter adjustment is performed by the implantable stimulator.

8. The method of claim 1 further comprising
providing at least one sensor;
using the at least one sensor to sense a physical condition; and
adjusting the stimulation parameters based on the sensed condition.

9. A method for treating a patient with angina pectoris, comprising:
providing a miniature implantable stimulator with at least one electrode and with a size and shape suitable for placement of the at least one electrode adjacent to a nerve;
implanting the at least one electrode near at least one tissue influencing the angina pectoris of the patient, which tissue is at least one of an intercostal nerve and an intercostal nerve branch;
providing operating power to the stimulator;
using an external appliance to transmit stimulation parameters to the stimulator;
receiving the stimulation parameters at the stimulator;
generating stimulation pulses in accordance with the stimulation parameters, which pulses are generated by the stimulator;
delivering stimulation pulses via the stimulator and the at least one electrode to the at least one of the intercostal nerves and intercostal nerve branches influencing angina pectoris as a treatment for angina pectoris.

10. The method of claim 9 wherein the at least one electrode is positioned on a lead, which lead is up to about 150 mm long.

11. The method of claim 9 further comprising generating and delivering excitatory stimulation pulses to at least one of the intercostal nerves and the intercostal nerve branches.

12. The method of claim 9 further comprising generating and delivering stimulation pulses of less than about 15 mA to at least one of the intercostal nerves and the intercostal nerve branches.

13. The method of claim 9 wherein the implantable stimulator further comprises at least one sensor and the method further comprises sensing at least one condition of the patient.

14. The method of claim 13 wherein the at least one sensed condition is used to adjust the stimulation parameters.

15. The method of claim 14 wherein the parameter adjustment is performed using the at least one external appliance.

16. The method of claim 14 wherein the parameter adjustment is performed by the implantable stimulator.

17. The method of claim 9 further comprising:
providing at least one sensor;
using the at least one sensor to sense a physical condition; and
adjusting the stimulation parameters based on the sensed condition.
